(19) United States
(12) Patent Application Publication
(10) Pub. No.: US 2022/0072181 A1
(43) Pub. Date: Mar. 10, 2022

(54) Air Treatment System For Cleaning Room Air

(71) Applicant: O3 Tech GmbH, Twistringen (DE)

(72) Inventors: Arend Brals, Ruinen (NL); Clemens Haskamp, Twistringen (DE)

(73) Assignee: O3 Tech GmbH, Twistringen (DE)

(21) Appl. No.: 17/468,902

(22) Filed: Sep. 8, 2021

(30) Foreign Application Priority Data
Sep. 8, 2020 (DE) 102020123367.1

(51) Int. Cl. A61L 9/04; A61L 9/22; A61L 9/20; F24F 8/40; F24F 8/30; F24F 8/22

(52) U.S. Cl. CPC A61L 9/04; A61L 9/22; A61L 9/20; F24F 8/40; F24F 2221/14; F24F 8/22; A61L 2209/15; A61L 2209/12; A61L 2209/111; F24F 8/30

(57) ABSTRACT

The present invention concerns an air treatment system for purifying room air, including an elongate carrier body, in particular a tube, of a predetermined carrier body length, wherein the carrier body is so designed that a passage extends with a passage diameter from an inlet side to an outlet side, and a ventilator unit adapted to convey room air through the passage of the carrier body from the inlet side to the outlet side with a predetermined air flow rate capacity, and an air treatment unit adapted to generate ozone in an ozone section within the passage in order to purify the room air being conveyed through the passage in the ozone section with the generated ozone and which is adapted to ionize the room air in an ionization section within the passage in order to purify the room air being conveyed through the passage in the ionization section by means of ionization.

The present invention concerns an air treatment system for purifying room air. In addition the present invention concerns a method of purifying air in a closed room.

Air treatment which can also be interpreted synonymously as air processing quite generally concerns the technical field of how air or constituents in the air are altered. The air treatment therefore includes not only removing constituents from the air like for example by filtering but it also includes adding constituents to the air.

The purification of air or air purification is a branch of air treatment and concerns the technical field of specifically removing unwanted constituents, for example chemical or organic constituents, from the air or reducing such constituents in the air.

Areas of application for air treatment systems are generally all regions in which unwanted organic or chemical constituents can be present in the air like for example in animal stalls, animal dissection establishments, fitness studios, festival tents, congress halls, sports venues, schools, warehouses, greenhouses, factories, veterinary clinics, hotels, restaurants, canteens or other larger commercial halls. There unwanted and unpleasant odors can occur or there may even be viruses, bacteria or fungi present in the air as suspended matter, which are pathogenic. Those pollutions can harm humans and animals and are therefore also referred to as noxious substances.

As a known proposal for air purification attention is directed to DE 10 2007 037 440. That document describes a highly complex and expensive technology in which inter alia an ozone sensor is also used, the measured values of which are in turn input into an open-loop and closed-loop control circuit in order thereby to control the ozone concentration of an ozone (O₃) generator.

Accordingly there is a need to remove unwanted chemical or organic constituents of all kind from the air or from the air in a closed room. Air in a closed room is referred to as room air.

The unwanted constituents in the air can be reduced or removed in different ways. For example systems are known which ionize air with an ionization device or mix the air with ozone with an ozone generator in order to remove unpleasant odors or organic substances from the air.

In that respect it is also known for entire rooms to be provided with an ozone atmosphere for a prolonged period of time as a one-off operation in order to kill off organic substances like for example bacteria, viruses or fungi. Disinfection is therefore effected with ozone which is also known as O₃. A problem with air purification with ozone is that ozone is to be avoided in an excessively high level of concentration in order to avoid influencing health wellness. Ozone however can be used to bind other noxious substances and remove them from the air. If a closed room is acted upon with an ozone atmosphere for air purification in a higher level of concentration that room should accordingly not be entered without protective measures. That is a disadvantage in particular for commercial enterprises as it involves operational stoppages and thus financial loss. In addition one-off air purification is not an ongoing solution to be able to operate rooms for the long term free from pollution or with a low level of pollution. After a one-off purification operation the pollution is admittedly briefly low but after some time impurities can occur afresh and the impurity of the air can become progressively greater to a degree which is once again problematical. A permanent solution for air purification is therefore desirable.

As air treatment systems are also provided for commercial applications in which rooms are mostly of a large volume there is also a general need that the system can be retro-fitted, can be inexpensively acquired, operates in a power-saving fashion and in that respect can purify or sterilize a sufficient volume of room air.

Therefore the object of the present invention is to address at least one of the above-mentioned problems, and in particular the invention seeks to provide a solution as to how relatively large amounts of room air are to be inexpensively, continuously and efficiently purified and pollutants in the air are to be kept as low as possible. At least however the invention seeks to propose an alternative to previously known structures or conceptionally establish or work out same.

According to the invention there is proposed an air treatment system for the purification of room air as set forth in claim 1.

Accordingly there is proposed an air treatment system which is intended to purify air, that is to say for example to remove or at least reduce unwanted pollutants from the air. Room air in that respect denotes air which is present in a closed room like for example air in a stall, a machine shop, a fitness studio or a hotel. Noxious substances can be organic pollutants of any kind like for example spores, fungi, bacteria or viruses and which are present in the form of suspended particles in the air. Noxious substances can also be chemical pollutants like for example ammonia, formic or butyric acid molecules. The air treatment system is thus intended to remove or at least reduce unwanted constituents in the air in a closed room.

For that purpose the air treatment system includes an elongate carrier body. Generally any form for the elongate carrier body is proposed, wherein there is an elongate carrier body if the length thereof is substantially greater than its diameter. In a particularly preferred embodiment the elongate carrier body is in the form of a tube, for example a round tube or a square tube. The carrier body is formed from a structurally strong material like for example metal, wood or a similar alternative material to serve as the carrier body or load-bearing structure. Electrical or mechanical components can be fixed to the carrier body like for example fixing elements for wall or ceiling fixing or further components of the air treatment system.

The carrier body is of a predetermined carrier body length which by way of example is in a range of 1 m through 20 m. The carrier body length accordingly denotes the overall length of the carrier body.

The carrier body in that case is of such a configuration that a passage having a passage diameter extends from an inlet side to an outlet side. The elongate carrier body is accordingly hollow and has a passage through which the air can flow and which extends from the inlet side to the outlet side. The section portion between the inlet side and the outlet side corresponds to the carrier body length. The passage can also be synonymously referred to as an air passage. It can extend in any form from the inlet side to the outlet side. The inlet side is a side of the carrier body at which air passes into the passage. The outlet side is a side of the carrier body at which air issues from the passage. A passage diameter refers to an inside diameter of the passage which for example is in a range of 0.2 m through 2 m. As a specific example the elongate carrier body is in the form of an aluminum round tube of an inside diameter of 0.37 m and a length between inlet side and outlet side of 5 m.

The air treatment system also includes a ventilator unit. That is adapted to convey room air through the passage of the carrier body from the inlet side to the outlet side with a predetermined air flow rate capacity. Accordingly the term ventilator unit denotes a technical device which is arranged at or in the carrier body and is adapted to convey room air through the passage of the carrier body from the inlet side to the outlet side. The ventilator unit is therefore arranged at the carrier body or is tightly connected thereto at least in terms of ventilation technology so that the ventilator unit conveys room air through the passage. For that purpose the ventilator unit can for example be mounted laterally directly to the carrier body or can be coupled to the carrier body in terms of ventilation technology with a connecting portion like for example a connecting hose. In addition the ventilator unit can also be inserted within the carrier body, for example in the form of an inserted axial ventilator. Examples of a ventilator unit are recirculation ventilators, fans, axial ventilators or blowers. In that case the air treatment system produces a constant air circulation with the ventilator unit so that the room air flows cyclically through the air treatment system and in so doing is purified.

The air treatment system also includes an air treatment unit. The air treatment unit is a technical device adapted to purify the room air and for that purpose is fixedly mounted for example to or in the carrier body. In that case the air treatment unit has two functions.

The air treatment unit is adapted to generate ozone which is written with the structural formula O₃ in an ozone section within the passage in order to purify the room air being conveyed through the passage in the ozone section with the generated ozone. In that case the ozone section is defined by a predetermined ozone maximum concentration and a predetermined ozone end concentration. The ozone concentration of the generated ozone decreases within the passage along the carrier body length in the direction of the outlet side to the ozone end concentration. The ozone maximum concentration describes a point or region within the passage of the carrier body, in which the concentration of the ozone is at a maximum and is for example 1.5 ppm (parts per million). That is approximately in a region in which the air treatment unit is arranged. The ozone end concentration describes a point or a region within the passage of the carrier body, at which there is a predetermined ozone concentration, for example 0.08 ppm ozone at the outlet side.

Accordingly the air treatment unit has the first function of acting as an ozone generator to generate ozone within the passage. In that respect the general operating principle of an ozone generator is that ozone is generated from oxygen in the air, which reacts as a strong oxidation agent with the air and thus promotes chemical degradation processes and can thus be used for odor removal and disinfection of the room air. How the ozone is generated in the tube can take place in different ways, for example by irradiation with a so-called ozone lamp or with an ozone injection in which ozone is injected from a storage container. Preferably the air treatment unit has an ozone lamp in order to generate the ozone in the ozone section within the passage. In other words the ozone is produced on site, within or in the direct proximity of the passage, and no harmful by-products occur, as the ozone reacts in a short time within the passage.

Due to the above-described property of the ozone as a highly reactive molecule the concentration of the ozone along the path through the passage to the outlet side decreases when room air is conveyed through the passage. The ozone basically binds the unwanted constituents from the room air, and accordingly the concentration of ozone decreases in the direction of the outlet side. On the basis of the ozone concentration it is thus possible to establish an ozone section which describes a lengthwise section within the passage of the carrier body and the starting and end points of which are defined by the ozone maximum concentration and the ozone end concentration. By way of example the ozone maximum concentration of 1.5 ppm can be established as the starting point of the ozone section and the ozone end concentration of 0.08 ppm can be established as an end point. The ozone concentration can be determined by measurement or calculation and can differ depending on the respective arrangement of the air treatment unit at or in the carrier body.

The air treatment unit is also adapted to ionize the room air in an ionization section within the passage in order to purify the room air being conveyed through the passage in the ionization section by means of ionization, wherein the ionization section is established by a predetermined first and second ionization intensity. The first and second ionization intensity can for example correspond to the two points within the passage, at which the air being conveyed begins and stops being ionized respectively.

Accordingly the air treatment unit has the second function of operating as an ionization device. The general operating principle of an ionization device is that air particles are ionized, which promote chemical degradation processes and can thus be used to remove odors and to disinfect the room air. The ionization results in so-called radicals, that is to say highly reactive atoms or molecules, which react with other air particles. The air can be ionized in different ways, for example by an ionizer with corona discharge or by irradiation with a UV lamp. Preferably the air treatment unit has a UV lamp in order to ionize the room air in an ionization section within the passage by means of UV irradiation.

Accordingly it is proposed that an ozone atmosphere is generated only within the carrier body or in the passage which is preferably in the form of a tube. The advantage there is that the entire room does not involve a harmful ozone atmosphere. In addition the ambient air can be recirculated by constant operation of the ventilator unit so that the room air circulates through the air treatment system a plurality of times. That continuously reduces the air pollution and also provides for deodorization. The air quality is thus progressively improved by continuous circulation.

In other words the use of a combined ozone and UV system is proposed for treatment and recirculation of the air, which contributes to reducing odors, for example by virtue of ammonia in the air. The proposed system can contribute to animal protection in particular in stalls for pigs, chicken, turkeys, cattle and sheep. In addition human health is less severely affected by bacteria, viruses, fungi or spores. It is therefore proposed to use an ozone and UV-C system which is used within the passage which can also be viewed as a reaction chamber, and with a ventilator unit like a recirculating ventilator which conveys room air through the passage. In the passage the ozone is then generated in combination with UV light and used for air treatment.

With the proposed system air is circulated at a predetermined frequency in the desired treatment room which is in a building or a stall so that the air is deodorized and disinfected during each passage therethrough. The odor and ammonia are then effectively broken down for example in the stall in which the animals are to be found. Accordingly deodorization and disinfection of the room air is made possible in a reliable fashion without harmful side effects like an excessively high level of ozone loading in the room.

The proposed air treatment system also attains the important effect that there is a constant circulation of air in the respective closed room or treatment room, and that circulation counteracts the causes of the air pollution. By virtue of the elongate carrier body that proposed air circulation does not contain any ozone as it reacts within the passage. Accordingly there is provided an air treatment system which can not only create a pleasant circulation in closed rooms but which also cyclically progressively frees the air from pollutants and noxious substances in the room air.

In that way the environment is considerably improved for humans and animals, and that also leads to fewer infections of the respiratory tracts in the animals. Ammonia emission is also reduced. The use of such an air treatment system also improves the function of an already installed air washer which is used for example in agricultural establishments and also has a positive effect on the use of chemicals, acids and lyes for the environment as it is possible to save on those.

By virtue of the structural configuration the system can also be used in small and large rooms, it can be retro-fitted in existing buildings, or it can be used for new structures. It permits installation without structural changes to the buildings or rooms. In addition the system has a very low power consumption and can be used without particular authorizations.

Preferably it is proposed that the predetermined carrier body length of the carrier body is established in dependence on a predetermined air conveyor time, wherein the air conveyor time describes a period of time that the air being conveyed through the passage requires to be conveyed from the inlet side to the outlet side by means of the ventilator unit. Particularly preferably it is proposed that the air conveyor time is established in dependence on the ozone end concentration. In a particularly preferred embodiment the carrier body length is selected to be so great that the ozone end concentration of the room air being conveyed occurs upon issue at the outlet side.

Accordingly it is proposed that the length of the carrier body is adapted to the ventilator unit and the air flow rate capacity or volume flow thereof is taken into consideration. In that respect the air flow rate capacity is given in m³h and is for example 12,500 m³/h. The air flow rate capacity is also referred to as the volume flow. In that respect the air conveyor time describes a period of time that the room air or a particle of the room air requires to be conveyed from the inlet side along the carrier body length to the outlet side. It is therefore proposed that the air conveyor time be selected to be such that the generated ozone completely reacts within the tube or the ozone end concentration occurs at the air outlet.

Preferably it is proposed that the predetermined carrier body length of the carrier body is established in dependence on the ozone section and the predetermined ozone end concentration occurs directly at the outlet side.

Accordingly it is proposed that the length of the carrier body is selected precisely to be of such a length that the predetermined ozone end concentration occurs precisely at the outlet side, that is to say at the end or at the air outlet of the carrier body. It is advantageous here that the length of the carrier body is at a maximum short but nonetheless the desired effect is achieved, namely that substantially no ozone issues at the outlet side. It is thus possible to save on material for the tube and the system can be of a compact structure.

It is preferably proposed that the passage diameter of the carrier body is larger than 0.2 m. In a particularly preferred embodiment the passage diameter is in a range of 0.2 m through 2 m, in particular the passage diameter being 0.37 m or 0.5 m.

Additionally or alternatively it is proposed that the carrier body length of the carrier body is larger than 1 m. In a particularly preferred embodiment the carrier body length is in a range of 1 m through 20 m, in particular the carrier body length being 5 m or 7.5 m.

Accordingly there is proposed a carrier body whose structural dimensions are designed for a high air throughput. Accordingly the air treatment system is particularly suitable for large rooms like stalls or halls.

It is preferably provided that the ozone maximum concentration is greater than 0.2 ppm. In a particularly preferred embodiment the ozone maximum concentration is in a range of 1 ppm through 2 ppm. In addition it is preferably proposed that the ozone concentration decreases along the carrier body length in the direction of the outlet to an ozone end concentration of less than 0.15 ppm. In a particularly preferred embodiment the ozone concentration decreases to a value in a range of 0.01 ppm through 0.15 ppm.

The abbreviation ppm stands for the usual English term “parts per million” and corresponds to a common quantitative flow rate. In the present case accordingly the unit describes the ozone concentration within the passage of the carrier body. Basically the ozone maximum concentration can be in a ratio to the power consumption of an ozone-generating device. In that respect the power consumption is higher, the higher the maximum ozone concentration. It was recognized in that respect that, with an ozone maximum concentration from 0.2 ppm, sufficient air purification can be achieved, and with an ozone maximum concentration in a range of 1 ppm through 2 ppm this involves efficient purification with at the same time a power-saving mode of operation of the ozone generator.

It is preferably proposed that the air treatment unit has an ozone lamp for continuously generating the ozone, the ozone lamp being adapted to generate ozone by means of electromagnetic radiation in a wavelength range of 175 nm through 195 nm, in particular with a wavelength of 185 nm.

Accordingly it is proposed that an ozone lamp is preferably used as an ozone generator for generating the ozone within the passage of the carrier body. An ozone lamp is a UV lamp which is characterised by a particular wavelength or a wavelength range in the UV range, more specifically a wavelength range of 175 nm through 195 nm. The ozone lamp has a lighting means for generating UV light in order to ionize the room air in the ionization section within the passage. In that respect UV is a common abbreviation for ultraviolet which describes electromagnetic radiation in the optical frequency range of light of shorter wavelengths than the light which is visible to a human being. Such an ozone layer is also suitable for continuous ozone generation. Generation of ozone is based on the fundamental principle that ozone is generated from the air by continuous irradiation with UV light in the given wavelength. In addition the ozone lamp ensures that the flow of air passing therethrough is permanently exposed to the given UV light to form the ozone.

It is preferably proposed that the air treatment unit has a UV lamp for continuous ionization of the room air, wherein the UV lamp is adapted to generate UV light by means of electromagnetic radiation in a wavelength range of 200 nm through 480 nm.

In a particularly preferred embodiment the UV lamp is adapted to generate UV-C light by means of electromagnetic radiation in a wavelength range of 200 nm through 280 nm, in particular with a wavelength of 254 nm. Accordingly in that wavelength range the UV lamp only emits UV-C light which is characterised by a wavelength range of 100 nm through 280 nm. In a particularly preferred embodiment the UV lamp is the form of a low-pressure UV lamp, for example a low-pressure mercury vapor lamp.

Accordingly it is proposed that a UV lamp is preferably used as the ionization device to ionize the room air in the ionization section within the passage. Accordingly room air is irradiated within the passage with a given ultraviolet radiation emitted by a UV lamp. The UV lamp accordingly has a lighting means. The method of air ionization is based on the fundamental principle that harmful substances or gases like volatile organic substances are oxidized and thus removed from the air by irradiation with light.

It is preferably proposed that the ventilator unit is arranged within the elongate carrier body or in terms of ventilation technology at the elongate carrier body at the inlet side and is preferably in the form of an axial ventilator. Accordingly there is proposed an inserted ventilator unit which is arranged at any location within the passage. In a particularly preferred embodiment the ventilator unit is arranged within the passage at the inlet side. That provides a robust and compact air treatment system.

It is preferably proposed that the ventilator unit has an air flow rate capacity of greater than 500 m³/h. In a particularly preferred embodiment the air flow rate capacity is in a range of 1,000 m³/h through 20,000 m³/h. That air flow rate is suitable in particular for large rooms in order for example to be able to implement a plurality of cycles of air circulation within a day. In addition it is possible to achieve continuous room air circulation with that flow rate in large rooms. The air flow rate capacity can be adapted to the respective location of use and in addition can be dependent on how frequently the air is to be circulated in relation to time, for example per day. In a particularly preferred embodiment the ventilator unit is driven by a drive device, preferably an electric motor, for example a constant-speed electric motor which is optimized for a predetermined rotary speed. Such a motor can be particularly energy-efficiently operated.

It is preferably proposed that the ventilator unit is adapted for permanent operation to convey room air cyclically through the air treatment system. In a particularly preferred embodiment the permanent operation is a continuous permanent operation, that is to say interruption-free operation of the ventilator unit so that a continuous steady air flow is permanently generated. In an alternative particularly preferred embodiment the permanent operation is a pulsating permanent operation, that is to say switching-on and switching-off operation or alternating operation of the ventilator unit so that a continuous steady air flow is generated cyclically for a predetermined and predeterminable period of time.

Additionally or alternatively it is proposed that the air treatment unit is designed for permanent operation to purify the room air cyclically within the passage. Similarly to the ventilator unit permanent operation of the air treatment unit in a particularly preferred embodiment is continuous permanent operation, that is to say interruption-free operation so that the air flowing through the passage is purified continuously and steadily with the air treatment unit. In a alternative particularly preferred embodiment the permanent operation is a pulsating permanent operation, that is to say a switching-on and switching-off mode of operation or alternating operation of the air treatment unit so that the air flowing through the passage is purified cyclically with the air treatment unit.

The permanent operation enables the room air to be circulated permanently in the room and the air to be cyclically purified. The continuous mode of operation is advantageous as a constant air flow is generated or constant air purification takes place. The pulsating mode of operation is more energy-saving.

It is preferably proposed that the elongate carrier body has fixing means for fixing the air treatment system to a wall and/or to a ceiling. In a particularly preferred embodiment the fixing means is in the form of a suspension means, for example a tube suspension means. In that way the entire air treatment system can be suspended to a ceiling or a wall, in which case this affords particularly efficient air purification if the air treatment system is fitted in an upper third of the room. As room air is known to heat up and as warm air rises upwardly and remains there warm air is more greatly polluted than cold air.

In addition the room air issuing at the outlet side is heated somewhat by the air purification operation so that the air treatment system additionally operates as a heating system.

In general it will be appreciated that, in designing or projecting the above-described air treatment system, for example the room volume of the closed room and the desired number of room air cycles per unit of time is to be taken into consideration. The air flow rate capacity of the ventilator unit is adapted thereto and in addition the structural dimensions of the carrier body like the passage diameter and the carrier body length are established in relation thereto. In addition the generation rate of the ozone of the air treatment unit or the ozone generator is taken into consideration for determining the length of the carrier body.

In addition according to the invention there is proposed a method of purifying air in a closed room. The method in that case includes the following steps:
providing an air treatment system including an elongate carrier body, namely a tube, having a predetermined carrier body length, wherein the carrier body is such that a passage with a passage diameter extends from an inlet side to an outlet side, a ventilator unit adapted to convey room air through the passage of the carrier body from the inlet side to the outlet side at a predetermined air flow rate capacity, and an air treatment unit adapted to generate ozone in an ozone section within the passage in order to purify the room air being conveyed through the passage in the ozone section with the generated ozone, wherein the ozone section is established by a predetermined ozone maximum concentration and a predetermined ozone end concentration, wherein the ozone concentration of the generated ozone decreases from the ozone maximum concentration along the carrier body length in the direction of the outlet side to the ozone end concentration, and the air treatment unit is adapted to ionize the room air in an ionization section within the passage in order to purify the room air being conveyed through the passage in the ionization section by means of ionization, wherein the ionization section is established by a predetermined first and second ionization intensity,
mounting the air treatment system to a ceiling of the closed room or in an upper region to a wall of the closed room in order to mount the air treatment system in a region with an accumulation in climate-control aspects of harmful gases, preferably in an upper third of the closed room, and
purifying the room air with the air treatment system.

Accordingly it is proposed that the air treatment system is fitted in the closed room where air purification is to be effected in a region which has an accumulation in climate-control aspects of harmful gases or harmful substances. That is for the most part in the upper third of the room. For that reason that region is preferably to be used. It was realized that the upper third is advantageous because the room air heats up, the warm air rises upwardly and the warm air is more heavily charged than cold air. In addition many production facilities use evaporators, in particular slaughter houses, which are also fitted relatively far up in the room and the evaporators can thus be firstly subjected to a purification effect.

Preferably it is proposed that the carrier body length is established in dependence on the predetermined ozone end concentration at the outlet side and in addition or alternatively is established in dependence on the air conveyor time of the ventilator unit.

It is preferably proposed that the air treatment system is designed in accordance with one of the embodiments. Accordingly it is proposed that the air treatment system is designed as described hereinbefore.

The advantage of the air treatment system according to the invention and also the method according to the invention for purifying air in relation to existing solutions, for example known from DE 10 2007 037 440, is that a simpler solution is created, which manages with less hardware, for example without an ozone sensor at the outlet of the tube (at any event such a sensor is not absolutely necessary) because with the solution according to the invention a predetermined carrier body length (tube length) of the carrier body is established or adapted in dependence on a predetermined air conveyor time, wherein the air conveyor time describes the period of time that the air being conveyed through the passage requires to be conveyed from the inlet side to the outlet side by means of the ventilator and the air conveyor time in turn is determined or adapted in dependence on the ozone end concentration at the outlet, that is to say the ozone end concentration at the outlet side of the carrier body (per tube) is always below a given quantity. If necessary therefore it is also possible by virtue of the solution according to the invention to manage with less hardware, for example without an ozone sensor and a control circuit as in DE 10 2007 037 440 in order nonetheless to implement optimum air purification. The solution according to the invention thus makes it possible to eliminate structural faults like for example wrong settings for the ozone sensor, the selection thereof, incorrect control implementation, defective switches and so forth.

If the air treatment unit has a UV lamp for continuous ionization of the room air, wherein the UV lamp is adapted to generate UVC light by means of electromagnetic radiation in a wavelength range of 200 nm through 280 nm, in particular with a wavelength of 254 nm, it is possible to achieve an air purification effect which is not attained by way of the state of the art.

A further advantage of the system according to the invention is also that, after manufacture, it can be easily installed in a room (typical plug and play solution) without further complicated setting operations having to be performed.

The first expert investigations and tests reveal that the use of the solution according to the invention leads to a reduction in germ carriers, viruses or the like in relation to the untreated control of on average 1.25 times the power of ten which corresponds to a purification result which hitherto was not yet achieved by comparable apparatuses.

The present invention will now be described in greater detail by way of example by means of embodiments with reference to the accompanying Figures, with the same references being used for identical or similar assemblies.

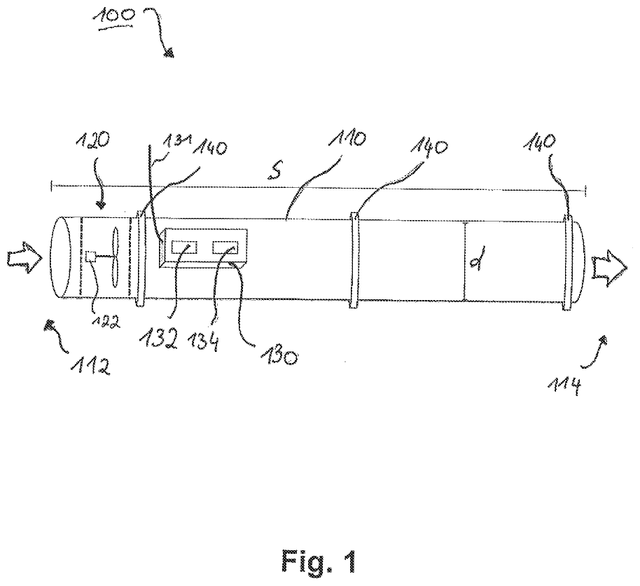

FIG. 1 diagrammatically shows a perspective view of an embodiment of an air treatment system,

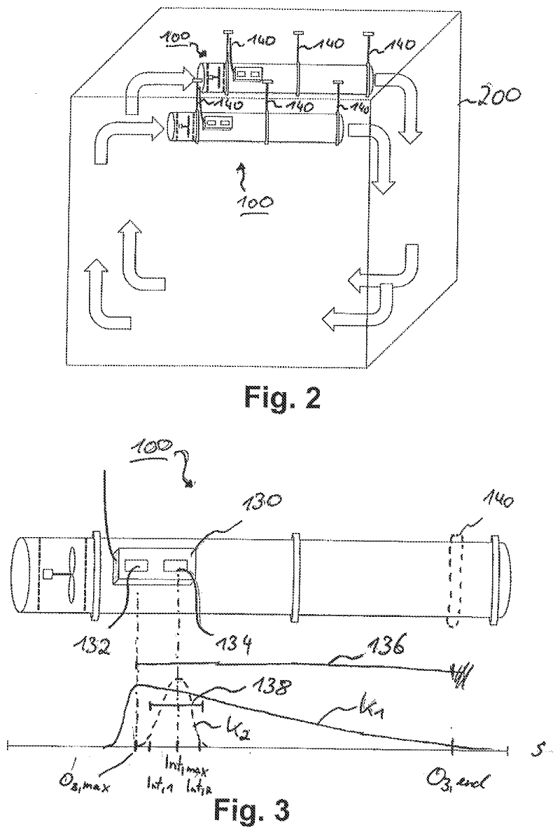

FIG. 2 shows a diagrammatic perspective view of two air treatment systems arranged in a closed room, and

FIG. 3 shows a diagrammatic view of characteristic curves along a carrier body length of an air treatment system in an embodiment.

FIG. 1 diagrammatically shows a perspective view of an air treatment system 100 for purifying room air in an embodiment, with which for example room air in a closed room can be purified, like for example the room 200 in FIG. 2.

The air treatment system 100 is provided for purifying room air. The room air is indicated by arrows in FIGS. 1 and 2, the direction thereof being intended to illustrate the flow direction. The air treatment system 100 has an elongate carrier body 110 in the form of a round tube which is of a passage diameter d which can also be interpreted as the inside diameter. The air or room air can flow through the tube 110 or round tube, more specifically from the inlet side 112 over the entire carrier body length s to the outlet side 114.

A ventilator unit 120 conveys the room air through the passage in the carrier body 110 from the inlet side 112 to the outlet side 114, more specifically with a predetermined air flow rate capacity. The flow rate capacity of the ventilator unit is established in that respect in dependence on the size of the room and the desired number of air circulations per unit of time. The ventilator unit 120 is in the form of an axial ventilator and is arranged within the tube 110. The ventilator unit 120 conveys a constant air flow through the passage that the tube forms. The ventilator unit 120 is designed for permanent operation. The ventilator unit 120 is driven by a drive device 122, for example an electric motor. That is optimized for a predetermined rotary speed for permanent operation in order to operate as energy-efficiently as possible. The air flow rate capacity is established, for example at 12,500 m³/h, by way of the rotary speed and the structural configuration of the ventilator unit.

An air treatment unit 130 generates ozone within the passage in order to purify the room air conveyed throught the passage with the generated ozone. In that respect the air treatment unit is only diagrammatically shown from the exterior and is illustrated in the form of a box which is fixedly mounted to the carrier body 110. The air treatment unit 130 has an ozone lamp 132, in which respect the rectangle 132 does not illustrate the ozone lamp but indicates the region where the ozone lamp 132 is arranged. The ozone lamp is arranged in the interior of the tube 110 (not shown) and emits UV light of a given wavelength, namely 185 nm. The air treatment unit 130 therefore generates ozone by means of the ozone lamp within the tube or in the passage. After being generated the ozone reacts with the room air flowing through the tube so that pollutant loadings in the air like organic loadings are bound and thus removed from the flowing air.

The air treatment unit 130 also ionizes the room air being conveyed through the passage by means of ionization. For that purpose in addition to the ozone lamp the air treatment unit 130 has a UV lamp 134, the rectangle 134 not showing the UV lamp but indicating the region where the UV lamp 134 is arranged. The UV lamp 134 is also arranged in the interior of the tube 110 (not shown) and emits UV-C light, although at a different wavelength from the ozone lamp 132. The UV lamp is in the form of a low-pressure UV mercury vapor lamp and irradiates the flowing air with a wavelength of about 254 nm, more specifically 253.7 nm. The pollutant loadings in the air are oxidized by the radiation so that they are reduced or decreased.

The air treatment unit 130 is accordingly to be viewed as a kind of combination device adapted to simultaneously implement two different kinds of air purification, namely with the ozone lamp 132 and with UV lamp 134. In that way it is possible to provide for particularly efficient air purification and in addition no ozone passes into the room as it reacts within the tube with the air being conveyed therein and is no longer present at all at the outlet side or is only still present there to a very low degree.

In addition a power supply 131 in FIG. 1 is used to supply the air treatment system 100 with power, like for example the ventilator unit 120 and the air treatment unit 130.

In addition the air treatment system 100 has three fixing elements 140 with which the tube 110 can be suspended from a ceiling or in an upper wall region.

FIG. 2 diagrammatically shows a perspective view of two air treatment systems 100 arranged in a closed room for the purification of room air, as shown for example in FIG. 1 or FIG. 3.

The air treatment systems 100 in this case are mounted to the ceiling of the room 200 with a plurality of fixing elements 140 which are in the form of a tube suspension means. It is thus provided that a plurality of air treatment systems 100 are also to be mounted in a room to increase the air purification rate. The two air treatment systems 100 provide for air circulation in the room 200, as indicated by the arrows in FIG. 2. The room air in the room 200 is correspondingly cyclically conveyed through the two air treatment systems 100. The air treatment systems 100 are arranged with tube suspension means 140 in a ceiling region or in the upper third of the room 200. This arrangement and the cyclic circulation of the room air correspondingly provides for ongoing air purification and efficient air purification without the ozone passing into the room, as described hereinbefore.

As a specific example the room 200 is of a room volume of 37,500 m³. The air flow rate capacity of the two ventilator units is 12,500 m³/h. That means that the complete room air is purified every 1.5 hours or, calculated per day, the complete room air is purified 16× per day.

FIG. 3 schematically shows a diagram with two schematic characteristic curves K1 and K2 and an air treatment system 100 as shown for example in FIG. 1 or FIG. 2.

The characteristic curve K1 describes an ozone concentration ([O₃ per m³] or ppm) in the room air being conveyed along the carrier body length s, that is to say along a path within the passage of the air treatment system 100. The air treatment system 100 has an air treatment unit 130 having an ozone lamp 132 and a UV lamp 134 as described for example hereinbefore with respect to FIGS. 1 and 2.

As can be seen from the characteristic curve K1 the ozone concentration within the tube forms a maximum which is referred to as the ozone maximum concentration (O_{3,max}). The maximum is formed approximately where the ozone lamp 132 which generates the ozone is arranged within the tube 110. When the air flows further along the carrier body length s the ozone concentration of the generated ozone steadily decreases within the tube as the ozone reacts with the air and is thereby progressively broken down. After some time or after a given travel distance the ozone concentration is reduced to the ozone end concentration (O_{3,end}). That can be established for example as being 0.08 ppm. The length section 136 which is between the ozone maximum concentration and the ozone end concentration is identified as the ozone section. It is in that longitudinal section that substantially a reaction of the ozone with the room air being conveyed occurs. The carrier body length s of the air treatment system 100 in that case is at least so long that the ozone end concentration (O_{3,end}) is reached. In order for example to save on tube material the carrier body or the tube length s can also coincide with the point at which the ozone end concentration O_{3,end} occurs. That is illustrated with the broken-line fixing element 140 in FIG. 3. In that way the tube is sufficiently long that the ozone is degraded, but it is at a maximum short.

The characteristic curve K2 describes an ionization intensity with which the room air being conveyed is ionized along the carrier body length s within the passage of the air treatment system 100. That can also be viewed as the radiation rate or radiation strength.

As can be seen from the characteristic curve K2 the ionization intensity forms within the tube a maximum which is identified as the maximum radiation intensity Int,ₘₐₓ. The maximum radiation intensity is formed approximately where the UV lamp 132 is arranged within the tube 100. The length section 138 which is between a first ionization intensity Int,1 and a second ionization intensity Int,2 is referred to as the ionization section as it is in that section that the air is substantially ionized or irradiated. When the room air being conveyed flows along the carrier body length s through the ionization section the pollution charges in the air are oxidized by the radiation so that they are reduced. The two points Int,1 and Int,2 are accordingly the points at which irradiation with UV light within the passage begins and ends.

### LIST OF REFERENCES

100 air treatment system

110 carrier body

112 inlet side

114 outlet side

120 ventilator unit

122 drive device

130 air treatment unit

131 power supply

132 ozone lamp

134 UV lamp

136 ozone section

138 ionization section

140 fixing element

d passage diameter

s carrier body length

O_{3,max} ozone maximum concentration

O_{3,end} ozone end concentration

1. An air treatment system (100) for purifying room air including
an elongate carrier body (110), namely a tube, having a predetermined carrier body length (s), wherein the carrier body is constructed so that a passage with a passage diameter (d) extends from an inlet side (112) to an outlet side (114),
a ventilator unit (120) adapted to convey room air through the passage of the carrier body from the inlet side to the outlet side at a predetermined air flow rate capacity, and
an air treatment unit (130) adapted to generate ozone (O₃) in an ozone section (136) within the passage in order to purify the room air being conveyed through the passage in the ozone section with the generated ozone, wherein the ozone section is established by a predetermined ozone maximum concentration (O_{3,max}) and a predetermined ozone end concentration (O_{3,end}), wherein the ozone concentration of the generated ozone decreases from the ozone maximum concentration (O_{3,max}) along the carrier body length in the direction of the outlet side to the ozone end concentration (O_{3,end}), and
the air treatment unit (130) is adapted to ionize the room air in an ionization section (138) within the passage in order to purify the room air being conveyed through the passage in the ionization section by means of ionization, wherein the ionization section is established by a predetermined first and second ionization intensity, and
the predetermined carrier body length of the carrier body is so established and/or designed in dependence on a predetermined air conveyor time, wherein the air conveyor time describes a period of time that the air being conveyed through the passage requires to be conveyed from the inlet side of the carrier body to the outlet side of the carrier body by means of the ventilator unit and the air conveyor time is established and/or adapted in dependence on the ozone end concentration and the predetermined ozone end concentration (O_{3,end}) 1 does not exceed a predetermined value, and/or
the air treatment unit (130) has a UV lamp (134) for continuous ionization of the room air, wherein the UV lamp is adapted to generate UVC light by means of electromagnetic radiation in a wavelength range of 200 nm through 280 nm, in particular in a wavelength of 254 nm.

2. An air treatment system as claimed in claim 1 characterised in that the predetermined carrier body length (s) of the carrier body (110) is established in dependence on the ozone section and the predetermined ozone end concentration (O_{3,end}) is present directly at the outlet side (114).

3. An air treatment system as claimed in claim 1, characterised in that
the passage diameter (d) of the carrier body is larger than 0.2 m, preferably in a range of 0.2 m through 2 m, in particular 0.37 m or 0.5 m, and/or
the carrier body length (s) of carrier body is larger than 1 m, preferably in a range of 1 m through 20 m, in particular 5 m or 7.5 m.

4. An air treatment system as claimed in claim 1, characterised in that
the ozone maximum concentration (O_{3,max}) is greater than 0.2 ppm, preferably in a range of 1 ppm through 2 ppm and the ozone concentration along the carrier body length in the direction of the outlet decreases to an ozone end concentration (O_{3,end}) of less than 0.15 ppm, preferably to an ozone end concentration in a range of 0.01 ppm through 0.15 ppm.

5. An air treatment system as claimed in claim 1, characterised in that
the air treatment unit (130) includes an ozone lamp (132) for continuously generating the ozone, wherein the ozone lamp being adapted to generate ozone by means of electromagnetic radiation in a wavelength range of 175 nm through 195 nm, in particular with a wavelength of 185 nm.

6. An air treatment system as claimed in claim 1, characterised in that
the ventilator unit (120) is arranged within the elongate carrier body (110) or at the elongate carrier body (110) at the inlet side and is preferably an axial ventilator.

7. An air treatment system as claimed in claim 1, characterised in that
the ventilator unit (120) has an air flow capacity which is greater than 500 m³/h, preferably in a range of 1,000 m³/h through 20,000 m³/h.

8. An air treatment system as claimed in claim 1, characterised in that
the ventilator unit (120) is adapted for permanent operation, in particular for continuous or pulsating permanent operation, and/or
the air treatment unit (130) is adapted for permanent operation, in particular for continuous or pulsating permanent operation to convey room air cyclically through the air treatment system.

9. An air treatment system as claimed in claim 1, characterised in that
the carrier body (110) has fixing means (140) for fixing the air treatment system to a wall and/or to a ceiling, wherein the fixing means is preferably in the form of a suspension means, in particular a tube suspension means.

10. A method of purifying air in a closed room including the steps:
providing an air treatment system (100) including an elongate carrier body (110), namely a tube, having a predetermined carrier body length, wherein the carrier body is constructed so that a passage with a passage diameter extends from an inlet side to an outlet side, a ventilator unit (120) adapted to convey room air through the passage of the carrier body from the inlet side to the outlet side at a predetermined air flow rate capacity, and an air treatment unit (130) adapted to generate ozone (O₃) in an ozone section within the passage in order to purify the room air being conveyed through the passage in the ozone section with the generated ozone, wherein the ozone section is established by a predetermined ozone maximum concentration (O_{3,max}) and a predetermined ozone end concentration (O_{3,end}), wherein the ozone concentration of the generated ozone decreases from the ozone maximum concentration along the carrier body length in the direction of the outlet side to the ozone end concentration, and the air treatment unit is adapted to ionize the room air in an ionization section within the passage in order to purify the room air being conveyed through the passage in the ionization section by means of ionization, wherein the ionization section is established by a predetermined first and second ionization intensity,
mounting the air treatment system to a ceiling of the closed room or in an upper region to a wall of the closed room in order to mount the air treatment system in a region with an accumulation in climate-control aspects of harmful gases or harmful substances, preferably in an upper third of the closed room, and
purifying the room air with the air treatment system.

11. A method as claimed in claim 10 characterised in that
the carrier body length is established in dependence on the predetermined ozone end concentration at the outlet side, and/or
is established in dependence on an air conveyor time of the ventilator unit.

12. A method as claimed in claim 10 characterised in that
the air treatment system is designed as claimed in claim 1.
